SEQUENCE LISTING (31 sequences)

SEQ ID NO: 1 - DNA, 1206 bp, Mus musculus
cgaagtcatggagcgctggccttggccgtcgggcggcgcctggctgctggtggctgcccgcgcgctgctgcagctgctgcgctcagacctgcgtctgggccgcccgttgttggcggcactggccctgctggctgctctcgactggctgtgccagcgcctactgcccccgccggcggcactcgtggtgctggctggtgctggctggatcgcgttgtcccgcctagcgcgccctcctcgcctgccggtggccactcgcgcggtgctcatcaccggttgtgacactggttttggcaaggagacagctaagaaactggatgccatgggcttcacggtgctggccacagtgttggatttgaatagccctggtgccctagaactgcgtgacctctgttctcctcgcctgaagctgctgcagatggatctgaccaaggcagaggacatcagccgtgttctggaaatcaccaaggcccacacggccagcactggcctgtggggtctggttaacaacgctggcctcaatatcgtagtggctgacgtggaactgtctccagtggcgactttccgcaagtgcatggaggtgaacttctttggtgcacttgagctgaccaagggcctcctgccactcttgcgtcactcgaggggacgtattgtgaccgttggcagcccagcaggagacatgccatacccctgcttggcagcctacggcacctccaaggcagcaatagcactgcttatggacacattcggctgtgagctgcttccctggggtatcaaggtcagcattatcaagcctggctgcttcaagacagatgcagtgactaatgtgaacctctgggagaaacgcaagcaactgctgctggccaacattcctagagagctgctccaggcctatggtgaagactacattgagcacgtgcacgggcagttcctgaattcactcagaatggcattgcctgaccttagcccagttgtagatgccatcatcgatgcattgctggcagctcagccacgaagccgctactacccaggccgtggcctggggctcatgtatttcatccaccactacctgccagagggcctgcgacgctgcttcctacagaacttctttatcaatcaccttctgccccgagcactgaggcccggccaacatggccctgctcctgcttaagacacacccttccccagtggcagctctgtgagccacaa

SEQ ID NO: 2 - DNA, 18 bp, Mus musculus
tggcaggaggtttccaca

SEQ ID NO: 3 - DNA, 23 bp, Mus musculus
agcagggccagtgccgccaacaa

SEQ ID NO: 4 - DNA, 11 bp, Homo sapiens
cctggcactac

SEQ ID NO: 5 - DNA, 11 bp, Bos taurus
cctggccttac

SEQ ID NO: 6 - DNA, 11 bp, Rattus rattus
cctggccctgt

SEQ ID NO: 7 - DNA, 11 bp, Oryctolagus cuniculus
cctggcgctac

SEQ ID NO: 8 - DNA, 11 bp, Equus caballus
cccagccctac

SEQ ID NO: 9 - DNA, 11 bp, Mus musculus
catggccctgc

SEQ ID NO: 10 - DNA, 10 bp, Mus musculus
catggcctgc

SEQ ID NO: 11 - protein, 27 aa, Homo sapiens
GQPGTTPPQDAAQDPNLSPGPSPAVAR

SEQ ID NO: 12 - protein, 26 aa, Bos taurus
GQPGLTSARDIAQDQGPRPDPSPTAQ

SEQ ID NO: 13 - protein, 22 aa, Rattus rattus
GQPGPVHDTTQDPNPSPTVSAL

SEQ ID NO: 14 - protein, 28 aa, Oryctolagus cuniculus
GQPGATPAPDTAQDNPNPNPDPSLVGAR

SEQ ID NO: 15 - protein, 27 aa, Equus caballus
GQPSPTPSQDAAQDPDSSPGTSPTAAR

SEQ ID NO: 16 - protein, 8 aa, Mus musculus
GQHGPAPA

SEQ ID NO: 17 - protein, 18 aa, Mus musculus
GQHGLLLLKTHPSPVAAL

SEQ ID NO: 18 - protein, 19 aa, Mus musculus
PGQHGLLLLKTHPSPVAAL

SEQ ID NO: 19 - protein, 9 aa, Mus musculus
PGQHGPAPA

SEQ ID NO: 20 - protein, 41 aa, Homo sapiens
AFFISHCLPRALQPGQPGTTPPQDAAQDPNLSPGPSPAVAR

SEQ ID NO: 21 - protein, 9 aa, Homo sapiens
AFFISHCLP

SEQ ID NO: 22 - DNA, 14 bp, Mus musculus
atcaccggttgtga

SEQ ID NO: 23 - DNA, 14 bp, Mus musculus
gcactggcctgtgg

SEQ ID NO: 24 - DNA, 14 bp, Mus musculus
cagcaggagacatg

SEQ ID NO: 25 - DNA, 16 bp, Mus musculus
ccaagacagatgcagt

SEQ ID NO: 26 - protein, 9 aa, Mus musculus
LPWGIKVSI

SEQ ID NO: 27 - protein, 8 aa, Mus musculus
LPWGIKMQ

SEQ ID NO: 28 - DNA, 18 bp, Mus musculus
ggggtatcaagatgcagt

SEQ ID NO: 29 - protein, 22 aa, Mus musculus
TRAVLITGCDTGFGKETAKKLD

SEQ ID NO: 30 - protein, 19 aa, Mus musculus
YGTSKAAIALLMDTFGCEL

SEQ ID NO: 31 - DNA, 1206 bp, Mus musculus
cgaagtcatggagcgctggccttggccgtcgggcggcgcctggctgctggtggctgcccgcgcgctgctgcagctgctgcgctcagacctgcgtctgggccgcccgttgttggcggcactggccctgctggctgctctcgactggctgtgccagcgcctgctgcccccgccggcggcactcgtggtgctggctggtgctggctggatcgcgttgtcccgcctagcgcgccctcctcgcctgccggtggccactcgcgcggtgctcatcaccggttgtgacactggttttggcaaggagacagctaagaaactggatgccatgggcttcacggtgctggccacagtgttggatttgaatagccctggtgccctagaactgcgtgacctctgttctcctcgcctgaagctgctgcagatggatctgaccaaggcagaggacatcagccgtgttctggaaatcaccaaggcccacacggccagcactggcctgtggggtctggttaacaacgctggcctcaatatcgtagtggctgacgtggaactgtctccagtggcgactttccgcaagtgcatggaggtgaacttctttggtgcacttgagctgaccaagggcctcctgccactcttgcgtcactcgaggggacgtattgtgaccgttggcagcccagcaggagacatgccatacccctgcttggcagcctacggcacctccaaggcagcaatagcactgcttatggacacattcggctgtgagctgcttccctggggtatcaaggtcagcattatcaagcctggctgcttcaagacagatgcagtgactaatgtgaacctctgggagaaacgcaagcaactgctgctggccaacattcctagagagctgctccaggcctatggtgaagactacattgagcacgtgcacgggcagttcctgaattcactcagaatggcattgcctgaccttagcccagttgtagatgccatcatcgatgcattgctggcagctcagccacgaagccgctactacccaggccgtggcctggggctcatgtatttcatccaccactacctgccagagggcctgcgacgctgcttcctacagaacttctttatcaatcaccttctgccccgagcactgaggcccggccaacatggccctgctcctgcttaagacacacccttccccagtggcagctctgtgagccacaa

(19) United States
(12) Patent Application Publication
(10) Pub. No.: US 2003/0221207 A1
(43) Pub. Date: Nov. 27, 2003

(54) Cardiac-specific 11beta hydroxysteroid dehydrogenase type 2 transgenic mice

(75) Inventors: Ellen G. McMahon, St. Louis, MO (US); Wenning Qin, Ballwin, MO (US); Joseph Goellner, St. Charles, MO (US); Amy E. Rudolph, St. Louis, MO (US)

Correspondence Address: BANNER & WITCOFF, 1001 G STREET N W, SUITE 1100, WASHINGTON, DC, 20001, US

(73) Assignee: Pharmacia Corporation, Global Patent Department, St. Louis, MO

(21) Appl. No.: 10/361,848

(22) Filed: Feb. 11, 2003

Related U.S. Application Data
(60) Provisional application No. 60/355,812, filed on Feb. 13, 2002.

(51) Int. Cl. A01K 67/027

(52) U.S. Cl. 800/18

(57) ABSTRACT

Five independent transgenic founder lines were created which have all developed cardiac hypertrophy and heart failure. The line with the most severe phenotype was analyzed in detail. Transgenic cardiac 11βHSD2 mRNA expression is increased 4,000 fold over non-transgenic mice and the expressed enzyme was found to possess catalytic activity. At five months of age transgenic mice had developed severe myocardial hypertrophy in the absence of an increase in blood pressure. Interstitial fibrosis in the left ventricle of transgenic mice was revealed by picrosirius red staining. The hearts of the mice were severely dilated and cardiomyocyte size was increased.

This application claims the benefit of provisional application Ser. No. 60/355,812 filed Feb. 13, 2002. The disclosure of the provisional application is expressly incorporated herein in its entirety.

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### BACKGROUND OF THE INVENTION

1. Field of the Invention

The invention relates to the field of cardio therapeutics. In particular, it relates to a model system for identifying and developing new drugs for treating cardiac failure.

2. Background of the Prior Art

Mineralcorticoid receptors (MR) are intracellular transcription factors which bind to specific regions of DNA (MRE-mineralcorticoid response elements) and increase the transcription of genes encoding specific aldosterone-induced proteins. Aldosterone has been shown to mediate maladaptive cardiac fibrosis and hypertrophy in heart failure by binding to mineralcorticoid receptors. When MR was first cloned and studied by Evans et al. (1987), a perplexing phenomenon was noted. The affinity of MR for the glucocorticoid cortisol was approximately 10-fold higher than the affinity for aldosterone. Since cortisol circulates at concentrations approximately 100-fold higher than aldosterone, MR should be overwhelmingly occupied by glucocorticoids rather than aldosterone. The mechanism that ensures aldosterone selectivity of MR in the distal nephron, distal colon, sweat and salivary gland is the co-expression of high levels of the enzyme 11β hydroxysteroid dehydrogenase type 2 (11βHSD2). This enzyme converts cortisol to its inactive 11-keto congener cortisone which is unable to bind to MR. Although MR does not distinguish between physiological glucocorticoids and aldosterone, 11βHSD2 can discriminate in that this enzyme cannot bind aldosterone. Therefore 11βHSD2 inactivates glucocorticoids in epithelial target tissues and allows aldosterone to bind and activate MR. Interestingly, in heart and brain, 11βHSD2 is absent and therefore MR in these tissues should be always occupied by glucocorticoids. Unlike in the kidney, where glucocorticoids are agonists of MR, in extraepithelial tissues, glucocorticoids appear to be anatagonists of MR.

There is a continuing need in the art for animal models of heart disease and for methods for identifying and developing new drugs for treating heart disease.

### BRIEF SUMMARY OF THE INVENTION

In a first embodiment of the invention a transgenic mouse is provided. The mouse expresses an increased amount of activity of enzyme 11-β hydroxysteroid dehydrogenase 2 (11βhsd2) in its heart relative to a non-transgenic isogenic mouse.

In a second embodiment of the invention a method is provided for screening test agents for the ability to mitigate cardiac fibrosis, cardiac hypertrophy, or cardiac failure. A test agent is administered to a transgenic mouse. The mouse expresses more enzyme activity of 11-β hydroxysteroid dehydrogenase 2 (11βhsd2) in its heart than a non-transgenic isogenic mouse. A biological phenomenon associated with cardiac fibrosis, cardiac hypertrophy, or cardiac failure is monitored in the mouse. A test agent that has a positive effect on the biological phenomenon is identified as a candidate drug for mitigating cardiac fibrosis, cardiac hypertrophy, or cardiac failure.

According to a third embodiment of the invention a method is provided for making a transgenic mouse. A DNA encoding 11βhsd2 is joined to a cardiac-specific promoter to form a construct. The construct is injected into pronuclei of fertilized mouse eggs to form transgenic eggs. The transgenic eggs are implanted into a pseudopregnant female mouse, and offspring are formed.

According to a fourth embodiment of the invention an isolated and purified nucleic acid is provided which encodes mouse 11-β hydroxysteroid dehydrogenase 2 (11βhsd2). The nucleic acid comprises the nucleotide sequence shown in SEQ ID NO: 1 or 31.

These and other embodiments of the invention which will be apparent to those of skill in the art upon reading the full disclosure provide the art with an excellent model system for studying cardiac dysfunction and for developing therapeutic approaches to treating cardiac dysfunction.

### BRIEF DESCRIPTION OF THE DRAWINGS

FIG. 1 shows the sequence of mouse 11βHSD2 cDNA isolated from kidney (SEQ ID NO: 1).

FIG. 2 shows a comparison of highly conserved amino acids of 11βHSD2 among different species, including human (SEQ ID NO: 4), Bos taurus (SEQ ID NO: 5), rat (SEQ ID NO: 6), rabbit (SEQ ID NO: 7), horse (SEQ ID NO: 8), and mouse (SEQ ID NO: 9 and 10).

FIG. 3 shows a comparison of consecutive amino acids of 11βHSD2 among different species, including human (SEQ ID NO: 11), Bos taurus (SEQ ID NO: 12), rat (SEQ ID NO: 13), rabbit (SEQ ID NO: 14), horse (SEQ ID NO: 15), and mouse (SEQ ID NO: 16 and 17) in the region of residues 379-386.

FIG. 4 shows a comparison between the published data (SEQ ID NO: 18) and the cloned mouse 11βHSD2 which was experimentally determined (SEQ ID NO: 19) and between a normal (SEQ ID NO: 20) and AME patient (SEQ ID NO: 21).

FIG. 5 shows a comparison between the mouse wild-type (SEQ ID NOS: 21-25) and splicing isoform of 11βHSD2 (SEQ ID NO: 26-27).

FIG. 6 shows the exon structure of wild type mouse 11βHSD2 including the coactivator binding domain and the active site (SEQ ID NOS: 28 and 29, respectively).

FIGS. 7A and 7B show the effect of Eplerenone in 11βhsd2 myocardio-specific transgenic mice. FIG. 7A shows echocardiogram data and FIG. 7B shows systolic blood pressure data.

FIG. 8 shows the transgenic construct of αMHC promoter and 11βhsd2.

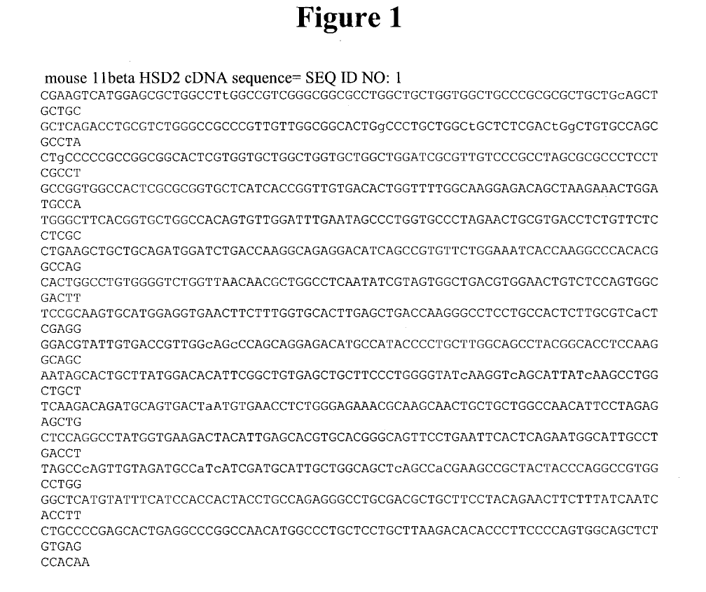

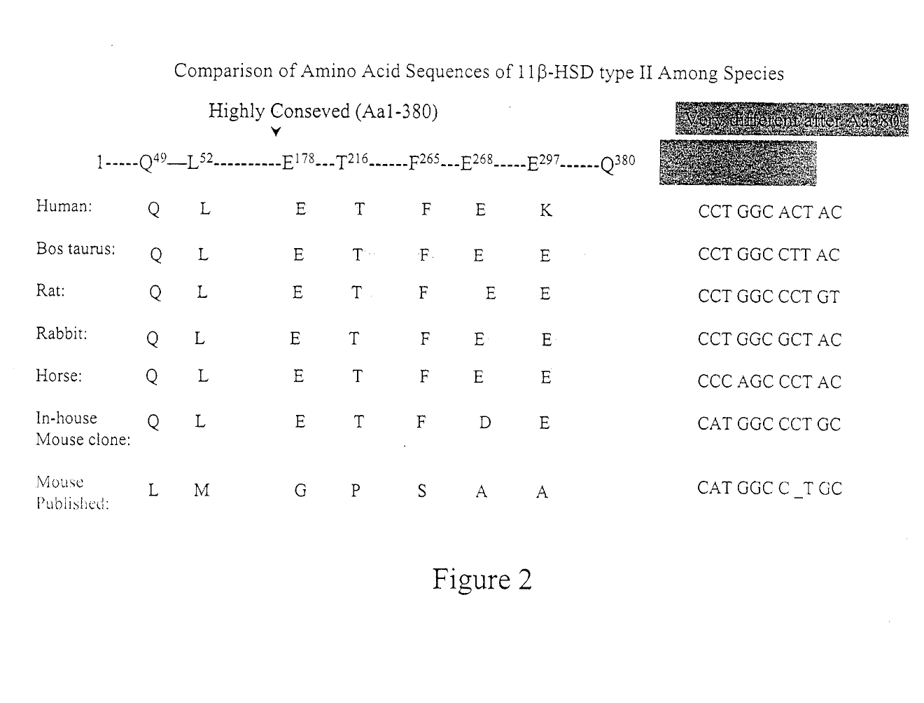

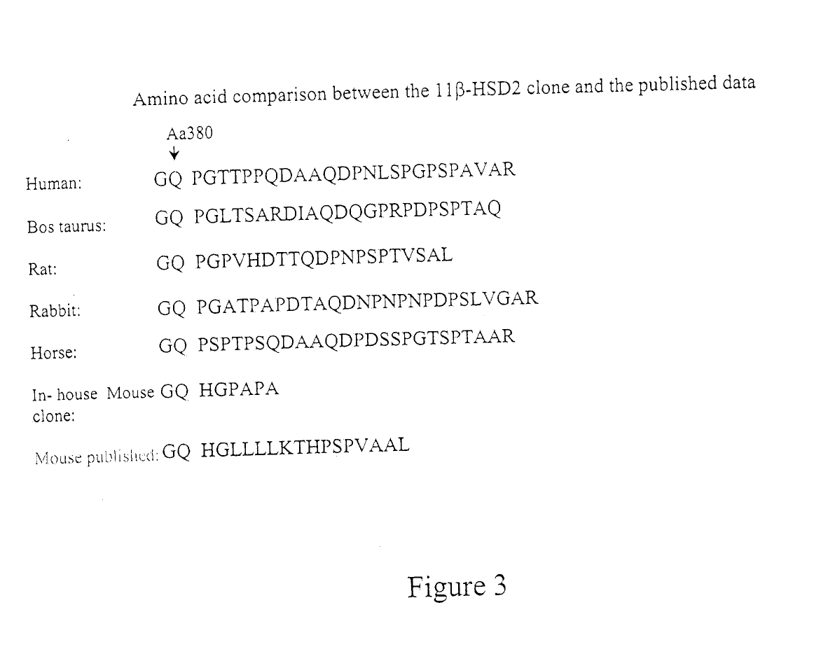

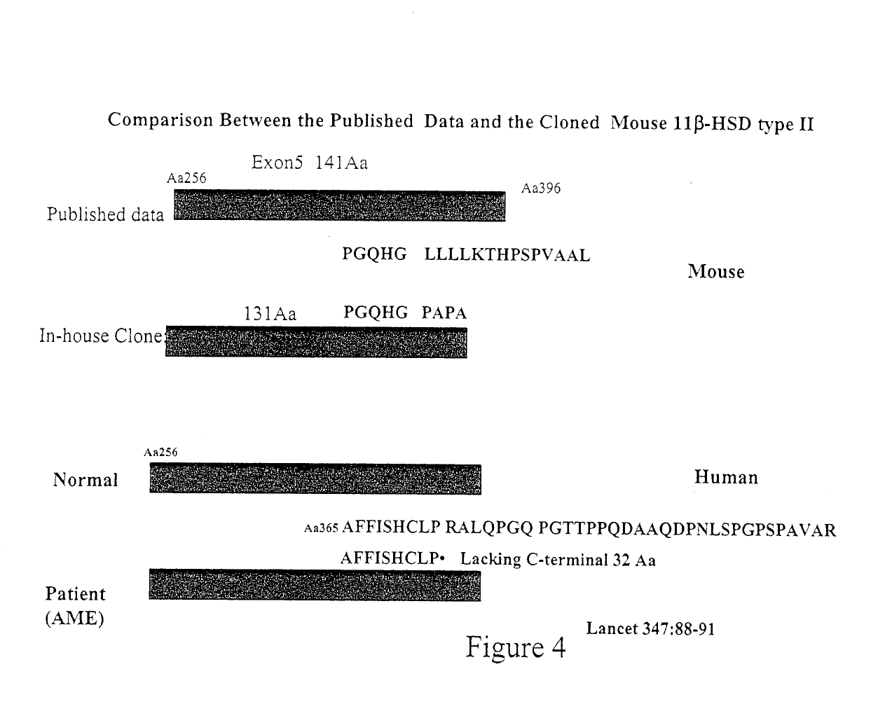

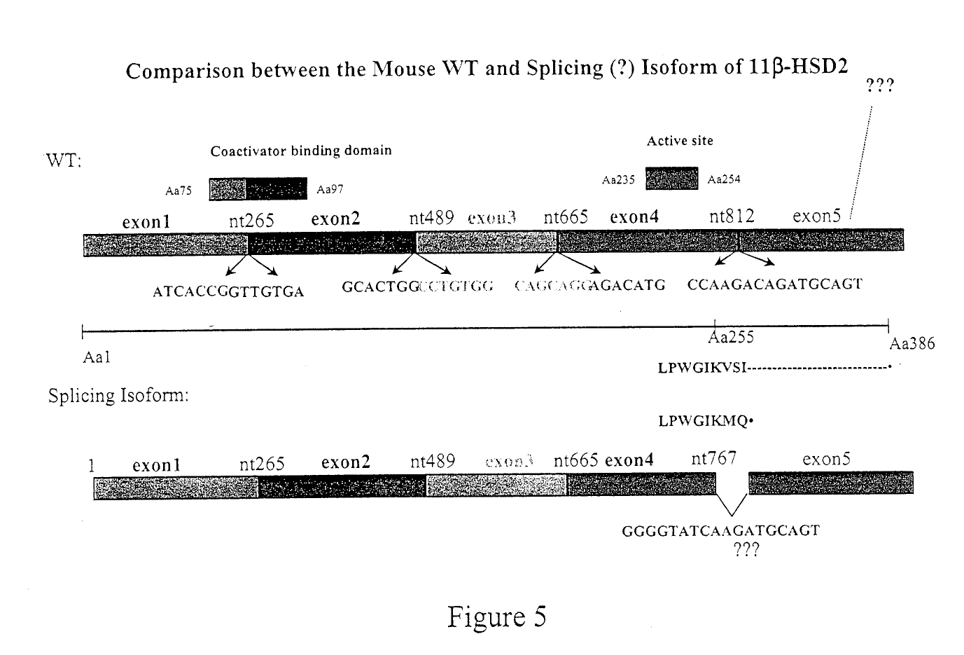

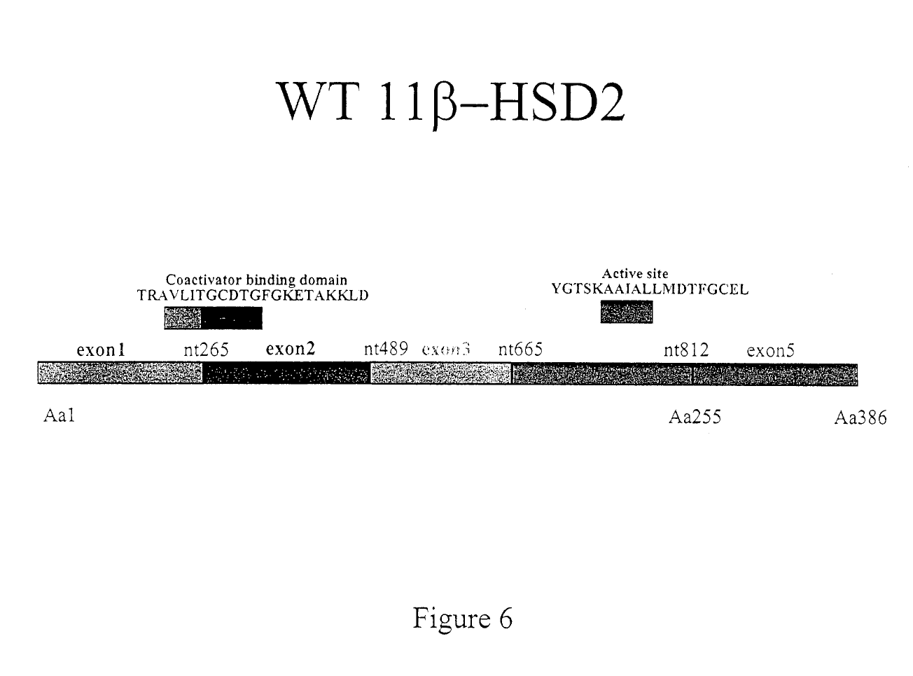

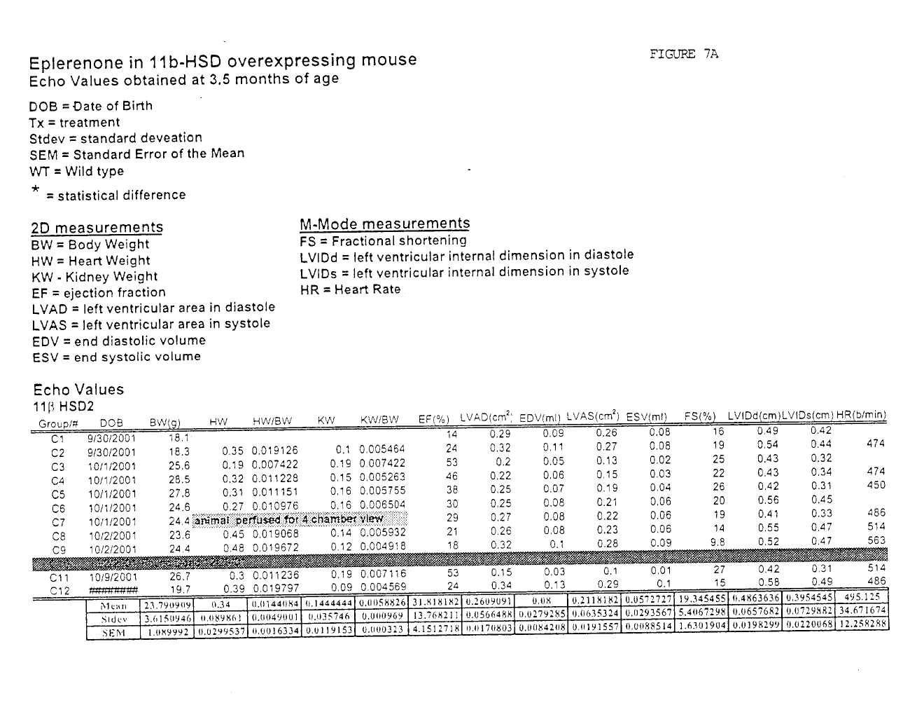

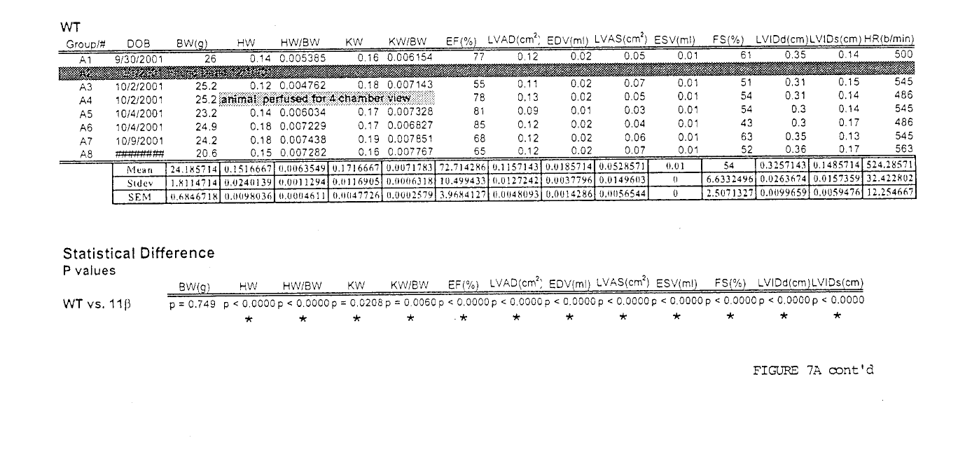

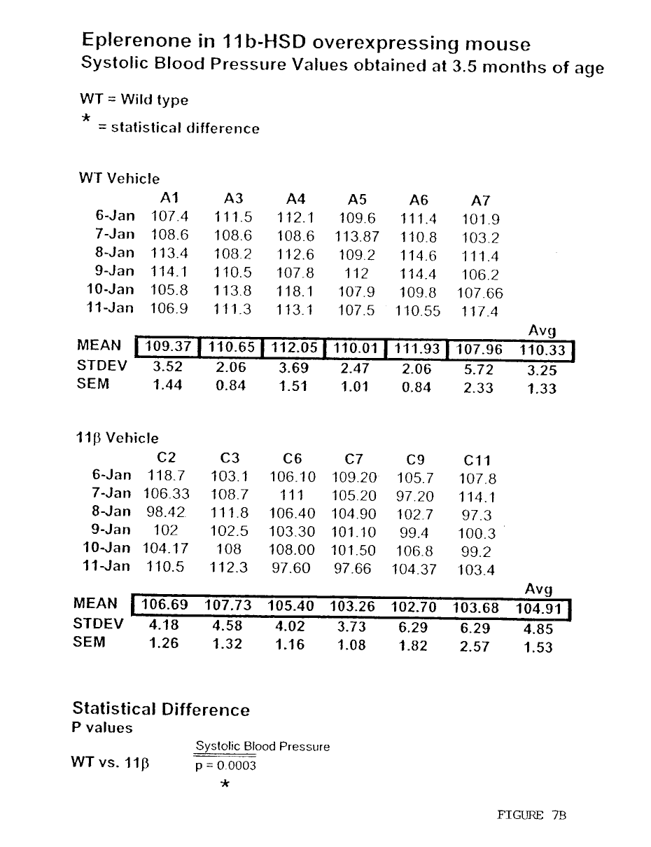

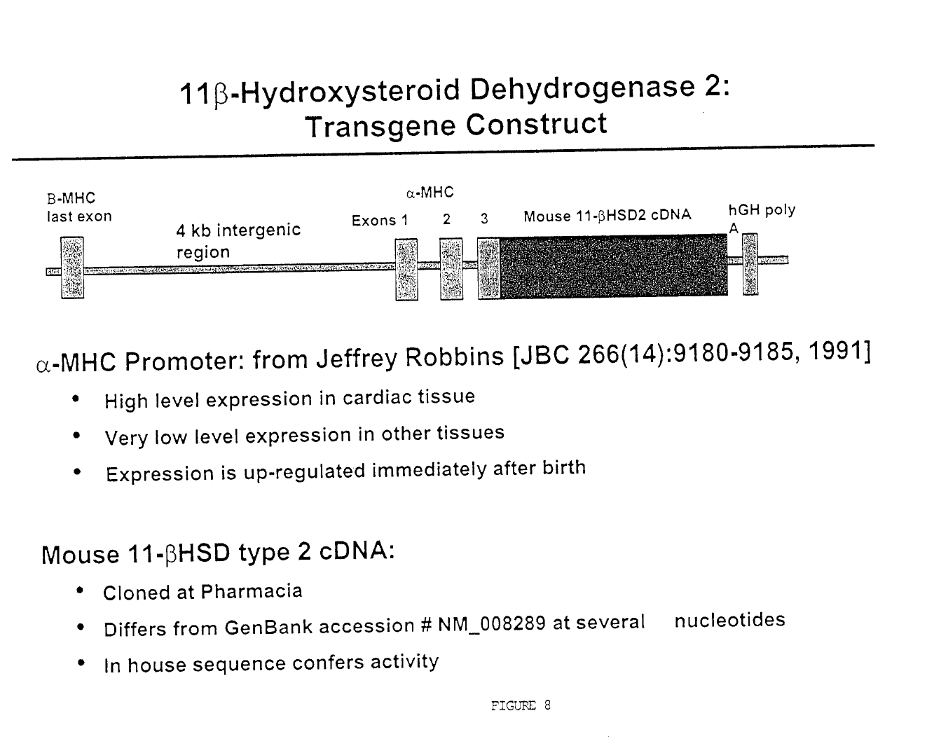

### DETAILED DESCRIPTION OF THE INVENTION

It is a discovery of the present inventors that a mouse which expresses more enzyme activity of 11-β hydroxysteroid dehydrogenase 2 (11βhsd2) in its heart than a non-transgenic isogenic mouse develops symptoms of cardiac disease, such as cardiac fibrosis, cardiac hypertrophy, and cardiac failure. Both structural and functional changes are observed in the transgenic mice. Such changes include, but are not limited to heart enlargement, early death, dilation of ventricles, collagen deposition in the heart, interstitial fibrosis, cardiomyocyte enlargement, thinning of ventricle walls, decreased ejection fraction, and decreased fractional shortening. The transgenic mouse thus represents an excellent model system for identifying and developing therapeutic agents for treating cardiac disease.

The transgenic mice of the invention specifically express 11βhsd2 in the heart, where it is typically not expressed or expressed at exceedingly low levels. The 11βhsd2 gene can be expressed in the cardiomyocytes and/or in other cardiac cells. Expression in the myocardium can also be useful. To achieve tissue specific expression it is desirable to use a cardiomyocyte-specific promoter. Suitable promoters include α-myosin heavy chain (αMHC) promoter, β-myosin heavy chain promoter, cardiac troponin C promoter, cardiac troponin T promoter, and cardiac troponin I promoter. Any promoter which provides cardiac-specific expression can be used. Cardiac-specific expression includes expression which is predominantly in the heart. Minor expression in other tissues can be tolerated. Desirably the promoter provides a level of expression which yields at least 50%, at least 100%, at least 200%, at least 500%, or at least 1000% more enzyme activity. However, any statistically significant increase in expression in the heart of the transgenic mouse as compared to the heart of an isogenic mouse not containing the transgene can be useful.

The 11βhsd2 coding sequence can be obtained from any mammal, including, but not limited to mouse, horse, chicken, human, rat, rabbit, and cow. A particularly useful coding sequence is that shown in SEQ ID NO:1. Polymorphic variants of these sequences can be used as well, without departing from the invention. It differs significantly from the sequence provided in GENBANK as accession no. NM_{—}008289. The coding sequence used can be in any usable form, including a genomic sequence or a cDNA sequence.

The transgenic mice of the present invention can be used to screen test agents for the ability to mitigate cardiac fibrosis, cardiac hypertrophy, or cardiac failure. Any test agent can be used. The test agent can be a single compound, a combination of defined compounds, or compositions containing multiple compounds, such as natural product extracts. The test agent can comprise known or novel compounds, those known to be useful for treating cardiac disease, or those previously unknown for such purposes. Exemplary agents known to be useful for treating cardiac disease that can be tested in combination with other agents include: angiotensin receptor blockers, calcium channel blockers, aldosterone antagonists, beta blockers, ACE inhibitors, diuretics, and digoxin. The test agents can be from compound libraries, from natural products libraries, synthetically made, or recombinantly made. The source of the test agent is not critical to the practice of the invention.

Transgenic mice which have been subjected to a test agent can be monitored for any biological phenomenon associated with cardiac fibrosis, cardiac hypertrophy, or cardiac failure. A test agent which is found to have a positive effect on the biological phenomenon is a candidate drug for mitigating cardiac fibrosis, cardiac hypertrophy, or cardiac failure. Those of skill in the art will recognize that the candidate drug will need to be further tested in other systems before they can be used in clinical practice. Those of skill in the art will further recognize that not all candidate drugs will pass all subsequent tests and be used successfully in clinical practice. Any further tests which can be employed for safety, efficacy, marketability, tolerability, etc. can be combined with the testing performed on the transgenic mice of the invention.

Biological phenomena which can be monitored in the transgenic mice include, without limitation, heart enlargement, inflammation, early death, dilation of ventricles, collagen deposition in the heart, interstitial fibrosis, ejection fraction, fractional shortening, cardiomyocyte enlargement, expression of a hypertrophic response gene, and thinning of ventricle walls. Any measure of structural heart damage or functional heart damage can be used to assess the effects of test agents on the transgenic mouse model of the invention. Parameters which can be measured include, without limitation, systolic blood pressure, left ventricular function, dilation and hypertrophy using echocardiographic techniques, 11βhsd2 enzyme activity in heart, kidney, aorta, and brain, histological characterization of left ventricle collagen content, fibrosis, quantitative PCR assessment of mRNA for 11βhsd2, ANP, MR, and MMP-9 and MMP-13 expression.

The nucleotide sequence encoding 11βhsd2 according to SEQ ID NO: 1 or 31 can be operably linked to a cardiomyocyte-specific promoter and/or a polyadenylylation signal. It can be in a self-replicating vector, such as a plasmid or virus, or it can be an isolated and purified DNA segment. Preferably the construct is integrated in the chromosome of the mouse. More preferably it is integrated in the endogenous mouse 11βhsd2 locus.

Additional transgenic mice similar to those which are described here can be made using techniques which are well known in the art. Briefly a DNA encoding 11βhsd2 is joined to a cardiac-specific promoter to form a construct. Typically this can be performed using ligase, but other methods are known in the art for joining two separate pieces of DNA and any such method can be used. The construct is injected into pronuclei of fertilized mouse eggs to form transgenic eggs. Again, any technique known in the art for accomplishing this goal can be used. The transgenic eggs are implanted into a pseudopregnant female mouse, and offspring are formed. The presence of the construct in an offspring can be tested by identifying a DNA sequence comprising a junction between DNA encoding 11βhsd2 and the cardiac-specific promoter. This can be performed using any technique known in the art, including but not limited to PCR, hybridization, oligonucleotides-specific ligation, sequencing, etc. Increased expression of 11βhsd2 in the offspring can be determined by measuring 11βhsd2-specific mRNA, e.g., using Northern blotting, RT-PCR, etc., by measuring 11βhsd2 protein, e.g., for example using Western blotting, or by measuring 11βhsd2 enzyme activity, e.g., using an enzyme assay.

Suitable promoters which can be used for cardiac specific expression include α-myosin heavy chain promoters (J. Biol. Chem.266: 9180-85, 1991) as well as those of β-myosin heavy chain promoter, cardiac troponin C promoter, cardiac troponin T promoter, and cardiac troponin I promoter. A polyadenylylation signal is also desirable at the 3′ end of the coding sequence of 11βhsd2. Any polyadenylylation signal can be used. A preferred signal is that from human growth hormone.

Enzyme activity can be measured using any technique known in the art. One suitable method employs thin layer chromatography and is described in Slight, S. H. et al., (1996) Journal of Molecular and Cellular Cardiology 28:781-787. Another suitable assay is described in Lombes, M. et al. (1995) Circulation 92: 175-182.

Several genes are known in the art to be expressed at an elevated level in cardiac hypertrophy. These genes are collectively known as the cardiac hypertrophy response genes. These genes include, without limitation, atrial natriuretic peptide (ANP) and β-myosin heavy chain (β-MHC). Expression of any one or more of these genes can be used as a biological phenomenon to monitor when evaluating the effects of test agents on the transgenic 11βHSD2 mice.

The transgenic 11βhsd2 mice of the present invention can be bred with other lines, whether transgenic or not. The other lines may be knock-out mice or classical mutants. The mice can be back-crossed or out-crossed to determine the effects of the transgene in different genetic backgrounds. One particularly preferred combination of traits is the combination of the transgenic cardiac-specific 11βhsd2 with an MR cardiac deletion. Such tissue specific deletions can be meade using the CRE-lox system.

### EXAMPLES

### Example 1

### Creating 11β HSD 2 Mice

The mouse 11β HSD2 cDNA was cloned by PCR (polymerase chain reaction) from mouse kidney total RNA and subcloned into pCR2.1 (Invitrogen, Calif.). The restriction fragment containing the entire coding sequence of the mouse 11βHSD2 cDNA was released from pCR2.1 and ligated into the Sal 1/Hind III sites of α-MHC Clone 26 kindly provided by Jeffrey Robbins. The transgenic construct containing the α-MHC promoter, 11βHSD2 cDNA and the hGH polyadenylation signal was released from the plasmid backbone by Not 1 digestion.

The transgenic construct was purified from agarose gel by Qiaex II kit (Qiagen, Calif.), resuspended in 10 mM Tris-HCl, 0.1 mM EDTA, pH 7.5, at 1 ng/μl, and injected into the pronuclei of the fertilized eggs of C57BL6 mice. Mice carrying the transgene were identified by the PCR reaction with the sense primer from the mouse α-MHC promoter (5′ TGGCAGGAGGTTTCCACA 3′; SEQ ID NO: 2) and the antisense primer from the mouse 11β HSD2 cDNA (5′ AGCAGGGCCAGTGCCGCCAACAA 3′; SEQ ID NO: 3), encompassing the junctional region between the promoter and the cDNA. Copy number between the founder lines was determined by Taqman analysis. The colony was maintained by littermate mating in the hemizygote state.

### Example 2

### Phenotypic Characterization

Five independent transgenic founder lines were created which have all developed cardiac hypertrophy and heart failure. The line with the most severe phenotype was analyzed in detail. Transgenic cardiac 11βHSD2 mRNA expression is increased 4,000 fold over non-transgenic mice and the expressed enzyme was found to possess catalytic activity. At five months of age transgenic mice had developed severe myocardial hypertrophy in the absence of an increase in blood pressure. Interstitial fibrosis in the left ventricle of transgenic mice was revealed by picrosirius red staining. The hearts of the mice were severely dilated and cardiomyocyte size was increased.

Preliminary histological examination indicated that both left and right ventricles are dilated and that LV and RV wall thickness is dramatically reduced in hearts from transgenic animals. Preliminary quantitation of collagen content indicates 10-fold higher collagen content in LV from transgenics compared to wild-type mice. These data suggest that when aldosterone is allowed to bind to MR in the heart, a significant deleterious effect is observed which eventually leads to decompensated heart failure and death. This may explain the stunning cardioprotective effect that occurs when aldosterone blockade is added on top of standard of care treatment for heart failure.

Starting at 4 weeks of age male transgenic mice were supplied with either chow containing eplerenone (approximate dose of 200 mg/kg/day) to eat or normal chow. After 2.5 months of treatment, mice in the untreated group showed deterioration of cardiac function. In contrast, myocardial function was significantly improved in the transgenic mice receiving eplerenone. Eplerenone treatment significantly attenuated the development of left ventricular dysfunction and heart failure in 11βhsd2 cardiac-specific transgenic mice.

### Example 3

### Ultrasound Echocardiography Acquisition and Analysis

Echocardiograms were acquired using a Sonos 5500 echocardiographic system equipped with a 15-MHz linear-array transducer (Agilent, Andover, Mass.). Images were obtained from mice lightly anesthetized with 1-2% isoflurane (AErrane; Baxter, Inc., Deerfield, Ill.) lying in the left lateral decubitus position. Care was taken to maintain adequate contact while avoiding excessive pressure on the chest wall. Two-dimensional parasternal long and short-axis images of the left ventricle were obtained. Two-dimensional targeted M-mode tracings were recorded from the parasternal short-axis view at the level of the papillary muscles at a sweep speed of 150 mm/s. All echocardiograms were recorded digitally on a rewritable magneto-optical disk. Measurements and calculations used are as follows: Left Ventricular End diastolic (EDV) and systolic (ESV) volumes were calculated via the method of discs from direct measurement systolic (LVAs) and diastolic (LVAd) areas. Ejection Fraction (EF) was calculated from systolic and diastolic volumes with the following formula: EF=(EDV−ESV)/EDV×100. Percent fractional shortening (FS) was calculated as follows: FS=(LVEDd−LVIDs)/LVIDd×100, where LVIDd and LVIDs are end diastolic and end-systolic LV internal dimensions, respectively. Heart rate (HR) was calculated by measuring the R-R interval in M-mode and using the formula: HR=60(sec/min)/R-R interval(sec/beat). All analysis measurements were performed using the leading-edge method according to the recommendations of the American Society for Echocardiography.

### Example 4

### Systolic Blood Pressure Acquisition

Training: Mice underwent a training session daily for 6 days to get accustomed to being in the mouse restrainers and tail cuffs for BP measurements using the Visitech BP tail cuff system, 2000 (Visitech Systems, Inc. Apex, N.C.). Each session included a set of 15 measurements for each mouse. Training was only considered to be complete when the average blood pressure was consistent for at least 2 days.

Procedure: Blood pressure was measured for groups of 4 mice simultaneously. Animals were placed on the heated platform (38° C. or 100° F.) with mouse restrainers and their tails in the tail cuff apparatus. A minimum of 5 preliminary cycles in each session was performed in order to allow the mice to warm up sufficiently to produce good blood flow in the tail. A set of 10 measurements was collected for every animal. Measurements obtained while the animal moved or during periods of weak signal were deleted from the set. All data obtained for individual mice were averaged for each day.

Experiment: Blood pressure measurements were performed once every month throughout the study. Each BP measurement started with a training period of 6 days continuing for another 6 days for data collection. Blood pressure from the last 6 days were recorded and used for data analysis. Averaging the data, one SBP value was obtained for each animal for that month. One SBP was obtained for a group by averaging the measures collected for each animal. Blood pressures are expressed as mean SBP±standard error mean.

### Example 5

### Description of Statistical Methodology

Treatment group means are compared based on a one-way analysis of variance (ANOVA) on the raw data. The means comparison method used is Least Significant Differences (LSD). The LSD means comparison procedure uses the pooled within group mean square error as the common estimate of the variance for all means comparisons. This is preferred to running several independent two-sample Student t-tests, since the variance estimate changes with each independent comparison. The calculations are the same as those used for the t-test, but the estimate of the variance is obtained from the one-way analysis of variance. Thus, the basis of comparison between each pair of means is consistent.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

We claim:

1. A transgenic mouse which expresses an increased amount of enzyme activity of 11-β hydroxysteroid dehydrogenase 2 (11βhsd2) in its heart relative to a non-transgenic isogenic mouse.

2. The transgenic mouse of claim 1 which expresses the enzyme in its cardiomyocytes.

3. The transgenic mouse of claim 1 wherein the enzyme is expressed under the transcriptional control of a cardiomyocyte-specific promoter.

4. The transgenic mouse of claim 1 wherein the enzyme is expressed under the transcriptional control of an α-myosin heavy chain promoter.

5. The transgenic mouse of claim 1 wherein the enzyme is expressed under the transcriptional control of a promoter selected from the group consisting of: β-myosin heavy chain promoter, cardiac troponin C promoter, cardiac troponin T promoter, and cardiac troponin I promoter.

6. The transgenic mouse of claim 1 which expresses at least 50% more enzyme activity.

7. The transgenic mouse of claim 1 which expresses at least 100% more enzyme activity.

8. The transgenic mouse of claim 1 which expresses at least 250% more enzyme activity.

9. The transgenic mouse of claim 1 which expresses at least 500% more enzyme activity.

10. The transgenic mouse of claim 1 which expresses at least 1000% more enzyme activity.

11. The transgenic mouse of claim 1 wherein the enzyme is a mouse enzyme.

12. The transgenic mouse of claim 1 wherein the enzyme is under the control of an α-myosin heavy chain promoter.

13. The transgenic mouse of claim 1 wherein the enzyme is expressed from a cDNA sequence.

14. The transgenic mouse of claim 1 wherein the enzyme is expressed from a sequence as shown in SEQ ID NO: 1 or 31.

15. A method of screening test agents for the ability to mitigate cardiac fibrosis, cardiac hypertrophy, or cardiac failure, comprising:
administering a test agent to a mouse according to claim 1;
monitoring a biological phenomenon associated with cardiac fibrosis, cardiac hypertrophy, or cardiac failure in the mouse, wherein a test agent which has a positive effect on the biological phenomenon is a candidate drug for mitigating cardiac fibrosis, cardiac hypertrophy, or cardiac failure.

16. The method of claim 15 wherein the biological phenomenon monitored is heart mass.

17. The method of claim 15 wherein the biological phenomenon monitored is early death.

18. The method of claim 15 wherein the biological phenomenon monitored is dilation of ventricles.

19. The method of claim 15 wherein the biological phenomenon monitored is collagen content of the heart.

20. The method of claim 15 wherein the biological phenomenon monitored is interstitial fibrosis.

21. The method of claim 15 wherein the biological phenomenon monitored is cardiomyocyte enlargement.

22. The method of claim 15 wherein the biological phenomenon monitored is thinning of ventricle walls.

23. The method of claim 15 wherein the test agent comprises a combination of compounds.

24. The method of claim 23 wherein the combination of compounds comprises at least one compound which is known for treating cardiac dysfunction.

25. The method of claim 23 wherein the combination of compounds comprises at least one compound selected from the group consisting of: angiotensin receptor blockers, calcium channel blockers, aldosterone antagonists, beta blockers, ACE inhibitors, diuretics, and digoxin.

26. The method of claim 15 wherein the biological phenomenon monitored is inflammation.

27. The method of claim 15 wherein the biological phenomenon monitored is cardiac function.

28. The method of claim 27 wherein the cardiac function monitored is ejection fraction.

29. The method of claim 27 wherein the cardiac function monitored is fractional shortening.

30. The method of claim 15 wherein the mouse comprises a sequence encoding 11βhsd2 according to SEQ ID NO: 1 or 31 operably linked to a cardiomyocyte-specific promoter.

31. The method of claim 15 wherein the biological phenomenon monitored is expression of a hypertrophic response gene.

32. A method of making a transgenic mouse comprising:
joining a DNA encoding 11βhsd2 to a cardiac-specific promoter to form a construct;
injecting the construct into pronuclei of fertilized mouse eggs to form transgenic eggs; and
implanting the transgenic eggs into a pseudopregnant female mouse, whereby offspring are formed.

33. The method of claim 32 further comprising:
confirming presence of the construct in an offspring by identifying a DNA sequence comprising a junction between DNA encoding 11βhsd2 and the cardiac-specific promoter.

34. The method of claim 32 further comprising:
confirming increased expression of 11βhsd2 in the offspring.

35. The method of claim 34 wherein increased expression is determined by measuring 11βhsd2-specific mRNA.

36. The method of claim 34 wherein increased expression is determined by measuring 11βhsd2 protein.

37. The method of claim 34 wherein increased expression is determined by measuring 11βhsd2 enzyme activity.

38. The method of claim 32 wherein the DNA encoding 11βhsd2 has a sequence according to SEQ ID NO: 1 or 31.
